(19) United States
(12) Patent Application Publication
(10) Pub. No.: US 2026/0295242 A1
(43) Pub. Date: Oct. 1, 2026

(54) FIXATION AND DELIVERY SYSTEMS AND METHODS FOR PACEMAKER DEVICES

(71) Applicant: PACESETTER, INC., Sylmar, CA (US)

(72) Inventor: Steve Chantasirivisal, Valencia, CA (US)

(21) Appl. No.: 19/629,346

(22) Filed: Mar. 26, 2026

Related U.S. Application Data
(60) Provisional application No. 63/780,682, filed on Mar. 31, 2025.

(51) Int. Cl. A61N 1/05

(52) U.S. Cl. CPC A61N 1/0573; A61N 2001/058

(57) ABSTRACT

A pacemaker device including a proximal end portion that includes a housing containing pacing electronics and battery material. The pacemaker device also includes a mount positioned at a distal end portion of the pacemaker device and a pliable assembly for fixating the pacemaker device to a tissue site. The pliable assembly is coupled to a mount and one or more electrodes electrically configured to receive one or more electrical signals from the tissue site and/or deliver one or more pacing signals to the tissue site.

### CROSS-REFERENCE TO RELATED APPLICATION

This application claims priority to U.S. Provisional Patent Application No. 63/780,682 filed on Mar. 31, 2025, which is incorporated by reference herein in its entirety.

### BACKGROUND

### a. Field

This disclosure relates generally to implantable pacemakers or biostimulators. More specifically, this disclosure relates to pacemaker devices with a header assembly and fixation elements.

### b. Background Art

Traditionally, pacemaker devices have actively anchored in cardiac tissue via active or passive fixation elements. Due to the complex anatomy of the heart and cardiac tissue, pacemaker devices must be carefully implanted to ensure correct electrical pulsing and reduce patient complications. Proper placement of the pacemaker devices reduces the risk of medical complications, for example, dislodgement and/or irregular heart activity.

Accordingly, there is an ongoing need to provide alternative fixation methods for pacemaker devices for implantation to ensure immediate and long-term therapeutic success.

### BRIEF SUMMARY

In one aspect, a pacemaker device adapted to be implanted in or attached to any desired location in a patient’s heart either endocranially, epicardially, or intramyocardially. The pacemaker device includes a proximal end portion that includes a housing defining a cavity for containing pacing electronics and battery material. The pacemaker device also includes a mount positioned at a distal end of the pacemaker device, a pliable assembly coupled to the mount, and one or more electrodes electrically configured to perform at least one receiving one or more electrical signals from a target tissue or delivering one or more pacing signals to the target tissue.

The pliable assembly may include one or more arms.

The one or more arms may include a plurality of slots operable to receive one or more anchors.

The one or more arms may be flexible and extend outwardly to contact the target tissue site.

The pliable assembly may include silicone material.

In another aspect, a method for positioning a pacemaker device adapted to be implanted in or attached to any desired location in a patient’s heart either endocranially, epicardially, or intramyocardially. The method includes The method includes guiding the pacemaker device to a target tissue site. The pacemaker device includes a mount and a pliable assembly coupled to the mount. The pliable assembly includes one or more anchors. The method also includes expanding the pliable assembly to contact the target tissue site in response to finding the target tissue site (e.g. stimulation site). The method further includes inserting the one or more anchors into the target tissue site, and delivering to the target tissue site, using the pliable assembly, one or more pacing signals.

### BRIEF DESCRIPTION OF THE DRAWINGS

Aspects of the present disclosure are set forth with particularity in the claims that follow. A better understanding of the features and advantages of the present disclosure will be obtained by reference to the following detailed description that sets forth illustrative systems and methods, in which the principles of the present disclosure are utilized, and the accompanying drawings of which:

FIG. 1 is a side view of an example pacemaker device.

FIG. 2 is a magnified view of an example proximal end of the pacemaker device of FIG. 1.

FIG. 3 is a diagrammatic medial-lateral cross section of a patient's heart illustrating a pacing system implanted in the patient’s heart.

FIG. 4 is a side view of an example of a pacemaker device including a distal end with a fixation attachment in accordance with the present disclosure.

FIG. 5 is a front view of the distal end of the pacemaker device of FIG. 4.

FIG. 6 is a side view of the pacemaker device of FIG. 4 in a catheter.

FIG. 7 is a first side view of the pacemaker device of FIG. 4 being implanted into a target tissue.

FIG. 8 is a second side view of the pacemaker device of FIG. 4 being implanted into a target tissue.

FIG. 9 is a first side view of the pacemaker device of FIG. 4 being retrieved from a target tissue.

FIG. 10 is a second side view of the pacemaker device of FIG. 4 being retrieved from a target tissue.

Corresponding and/or like reference numerals used throughout the drawings indicate corresponding and/or like features and elements.

### DETAILED DESCRIPTION

The systems and methods described herein relate to pacemaker systems and methods that leverage pacemaker geometry and fixation elements for a pacing device (also referred herein as to pacemaker device or biostimulator) suitable for use in a diverse patient population, which increases access to life sustaining pacing technology for a vulnerable patient population. This approach may reduce the risk of dislodgement and help patients with cardiac issues.

The fixation approach described herein may reduce the risk of fixation displacement and simplify management and help reduce fixation displacement due to unique heart anatomical factors. In the systems and methods described herein, a pacing device includes a proximal end portion and distal end portion. The distal end portion includes a header assembly that extends distally from a distal end of the proximal end portion and is further coupled to a mount which includes one or more fixation elements and one or more electrodes. The proximal end portion may be configured to provide a compact biostimulator volume which contains an energy source and circuitry. The one or more fixation elements extend from the mount, which is configured for implanting into a cardiac chamber of the patient’s heart, and the distal electrode(s), which is configured to sense and/or deliver electrical signals to a target tissue. In some cases, at least one of the one or more fixation elements may also operate as an electrode and may be implanted into a chamber of the patient’s heart. In other cases, at least one of the one or more fixation elements may be implanted into the epicardium at a sufficient depth to reach the myocardium for sensing and/or delivering electrical signals. The compact size of the pacemaker device enables a high degree of versatility with respect to the locations where the pacemaker device is positioned.

Advantages provided by the systems and methods described herein include, but are not limited to: 1) the pacemaker device being suitably sized for multiple patient populations; 2) the pacemaker device being sized to enable device placement in locations that may aid in lead survivability or longevity; 3) the pacemaker device being placed more accurately at an angle for more precise placement when the internal anatomy of a patient is difficult to navigate; 4) the pacemaker device being used in conjunction with an implant tool and a thoracoscopeic (thorascopic camera) subxiphoid approach, thereby limiting or eliminating the need for placement via thoracotomy or other high-risk procedures; and 5) the pacemaker device including a fixation mechanism including passive pliable anchors that reduce or eliminate challenges due to the atrial wall being thin that may results in fixation mechanisms protruding through the atrial wall and into the pericardium causing pericardium pinning.

Before beginning a detailed discussion of the pacemaker device of the present disclosure, a general overview of an example pacemaker device is provided as follows.

### A. Overview of Pacemaker Devices

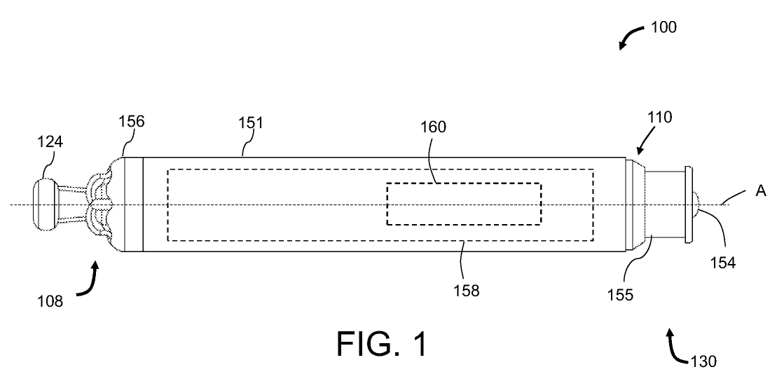

FIG. 1 illustrates an example biostimulator or pacemaker device 100 (e.g., a leadless or leaded pacemaker device) configured for implanting at an implant site (e.g., within an atrium or a ventricle of a patient’s heart, or an epicardial region of the patient’s heart). Pacemaker device 100 may communicate by conducted communication, representing a substantial departure from conventional pacing systems. Pacemaker device 100 may perform cardiac pacing that has many of the advantages of conventional pacemaker devices while extending performance, functionality, and operating characteristics.

In some implementations, pacemaker device 100 provides cardiac pacing without a pulse generator located in the pectoral region or abdomen, without an electrode-lead separate from the pulse generator, without a communication coil or antenna, and without an additional requirement of battery power for transmitted communication.

As may be understood from FIG. 1, pacemaker device 100 may have two or more electrodes 154, 156 located within, on, or near a housing 151, for sensing and/or delivering electrical activity at the muscle of the cardiac chamber and optionally for sensing electrical activity from the muscle, and for bidirectional communication with at least one other device within or outside the body. For example, housing 151 may have a longitudinal axis A, and distal electrode 154 may be a distal pacing electrode mounted on the housing along longitudinal axis A. Housing 151 may act as proximal electrode 156 (e.g., a ring electrode). Ring electrode 156 may be the anode and distal electrode 154 may be the cathode. In some implementations, housing 151 may include a conductive material such as titanium, 316L stainless steel, or other similar materials, and is partially coated with an insulating or dielectric (e.g., polymeric) coating, with the uncoated region of housing 151 defining proximal electrode 156 that is proximal to distal electrode 154.

Housing 151 may be hermetic and contain, within a sealed cavity 158, a primary battery to provide power for pacing, sensing, and communication, which may include, for example bidirectional communication. Housing 151 may also contain, within sealed cavity 158, circuits for sensing cardiac activity from electrodes 154, 156. Housing 151 may contain circuits for receiving information from at least one other device via electrodes 154, 156 and contains circuits for generating electrical signals for delivery and/or receiving electrical signals via the electrodes 154, 156. Housing 151 may contain circuits for transmitting information to at least one other device via the electrodes 154, 156 and may optionally contain circuits for monitoring device health. Housing 151 may contain circuits for controlling these operations in a predetermined manner.

Pacemaker device 100 includes a header assembly 110 that may be mounted on a distal end portion 130 of housing 151 along longitudinal axis A. Header assembly 110 may include an electrical feedthrough assembly including an electrical feedthrough (not shown) and distal electrode 154, (e.g., a pacing tip). Example electric feedthrough assemblies are described, for example, in U.S. Pat. No. 11,247,059, issued on Feb. 15, 2022, entitled “Biostimulator Having Flexible Circuit Assembly,” the disclosure of which is incorporated by reference in its entirety. Header assembly 110 may include a mount 155 mounted on the electrical feedthrough assembly around longitudinal axis A. A fixation element (e.g., the one or more anchors or tines) is mounted on mount 155 along longitudinal axis A. The assembled components of pacemaker device 100 may provide a distal region that attaches to a target tissue, e.g., via engagement of the fixation element with the target tissue. The distal region may deliver a pacing impulse to the target tissue, e.g., via distal electrode 154 that is held against the target tissue.

As described above, hermetic housing 151 defines sealed cavity 158 that may contain electronics and circuitry. These may be disposed in an electronics compartment 160 of sealed cavity 158. More particularly, housing 151 may have a housing wall, e.g., a cylindrical wall, laterally surrounding electronics compartment 160. In some cases, the housing wall has an inner surface extending around electronics compartment 160 on longitudinal axis A. The housing wall may include a conductive, biocompatible, inert, and anodically safe material such as titanium, 316L stainless steel, or other similar materials, to laterally enclose electronics compartment 160. Electronics compartment 160 may be axially enclosed at its proximal end by the battery. More particularly, a distal surface or face of the battery may define the proximal end of electronics compartment 160. Electronics compartment 160 may be axially enclosed at a distal end by header assembly 110. More particularly, a proximal surface of header assembly 110 may define the distal end of electronics compartment 160. Housing 151 may be attached, e.g., threaded, adhered, or welded, to header assembly 110 and the battery. Accordingly, electronics compartment 160 may be contained between the battery, the inner surface of housing 151, and header assembly 110.

Advantageously, housing 151 is relatively compact and has a relatively small footprint, which enables pacemaker device 100 to be placed within the body of the patient inside the heart and/or in proximity to the heart. The relatively small size of pacemaker device 100 provides a relatively high degree versatility with respect to the locations in the patient within which pacemaker device 100 may be placed in within the heart. In this regard, the location at which pacemaker device 100 is not limited and may vary depending on various factors including intended application, size of the patient, preferences of the physician, among other considerations.

In an implementation, as described, for example in U.S. Pat. No. 11,247,059, issued on Feb. 15, 2022, entitled “Biostimulator Having Flexible Circuit Assembly”, which is incorporated by reference in its entirety, a flexible circuit assembly is contained within electronics compartment 160. The flexible circuit assembly may include a flexible substrate having one or more electronic components mounted on a flexible substrate. For example, the flexible circuit assembly may include one or more passive electronic components, e.g., capacitors, and one or more active electronic components, e.g., processors. The electronic components may be interconnected by electrical traces, vias, or other electrical connectors Electronics compartment 160 may also include one or more electrical connectors, e.g., socket and pin connectors or metallized contact pads, to connect to the battery and the electrical feedthrough assembly. For example, the electrical connector may be a socket connector or a metallized pad to receive and/or connect to an electrode pin or a terminal pin.

The electrical connectors of the flexible circuit assembly may be accidentally short-circuited to other conductive components of pacemaker device 100 such as housing 151 or battery. To reduce the likelihood of such an event, pacemaker device 100 may incorporate components to electrically insulate and/or protect the flexible circuit assembly components from short-circuiting. For example, pacemaker device may include an end insulator (not shown) that includes a planar structure to form a wall between the flexible circuit assembly and the energy source. Suitably, the end insulator may separate the battery, and more particularly an enclosure of the battery, from the flexible circuit assembly. Pacemaker device 100 may also include a wall insulator that separates the flexible circuit assembly from the inner surface of housing 151. It will be appreciated that the flexible substrate of the flexible circuit assembly may provide sufficient insulation and separation from housing 151 and the battery, and thus, the end insulator and the wall insulator are optional.

Pacemaker 100 also includes a proximal end portion 108 that may contain a proximal attachment feature 124 (e.g., a button and stem) configured for engaging a docking cap or key of a delivery system (e.g., a catheter). Examples of attachment features suitable for delivery and retrieval of pacemaker device 100 are described, for example, in U.S. Pat. No. 11,141,597, issued on Oct. 12, 2021, entitled “Leadless Pacemaker Having Attachment Feature”, the disclosure of which is incorporated by reference in its entirety. In other implementations, the delivery system includes clips designed to match the shape of a feature on pacemaker device 100 and apply torque to screw the active engaging mechanism into the tissue.

Pacemaker device 100 may be adapted for delivery and implantation into tissue in the human body. As described above, pacemaker device 100 may be adapted for implantation adjacent to heart tissue on the inside or outside wall of a cardiac chamber, using two or more electrodes located on or within the housing of pacemaker device 100 for pacing the cardiac chamber upon receiving a triggering signal from at least one other device within the body.

Pacemakers or other biostimulators are typically fixed to an intracardial implant site by an actively engaging mechanism or a fixation mechanism such as a screw or helical member that screws into the myocardium. Examples of such biostimulators are described in the following publications, the disclosures of which are incorporated by reference in their entirety: (1) U.S. Pat. No. 8,457,742, issued on Jun. 4, 2013, entitled “Leadless Cardiac Pacemaker System For Usage In Combination With An Implantable Cardioverter-Defibrillator”; (2) U.S. Pat. No. 9,358,400, issued on Jun. 7, 2016, entitled “Leadless Cardiac Pacemaker”; (3) U.S. Pat. No. 9,216,298, issued on Dec. 22, 2015, entitled “Leadless Cardiac Pacemaker System with Conductive Communication”; (4) U.S. Pat. No. 8,352,025, issued on Jan. 8, 2013, entitled “Leadless Cardiac Pacemaker Triggered by Conductive Communication”; (5) U.S. Pat. No. 7,937,148, issued on May 3, 2011, entitled “Rate Responsive Leadless Cardiac Pacemaker”; (6) U.S. Pat. No. 7,945,333, issued on May 17, 2011, entitled “Programmer for Biostimulator System”; (7) U.S. Pat. No. 8,010,209, issued on Aug. 30, 2011, entitled “Delivery System for Implantable Biostimulator”; (8) International Application No. PCT/US2006/040564, filed on Oct. 13, 2006, entitled “Leadless Cardiac Pacemaker and System” and published as WO07047681A2 on Apr. 26, 2007; and (9) U.S. Pat. No. 11,247,059, issued on Feb. 15, 2022, entitled “Biostimulator Having Flexible Circuit assembly.”

In addition to the fixation mechanism, such as a helix, some biostimulators may further include additional or different fixation mechanism to provide another feature for keeping the biostimulator in place within the body. These fixation mechanisms may be either active (e.g., the fixation mechanism may actively engage tissue, either within or outside the heart), or may be passive (e.g., the fixation mechanism is not attached to tissue but rather prevents the biostimulator from moving around in the body in the case of accidental detachment). Further details on these fixation mechanisms may be found in U.S. Pat. No. 8,527,068, issued on Sep. 3, 2013, entitled “Leadless Cardiac Pacemaker with Secondary Fixation Capability”, the disclosure of which is incorporated by reference in its entirety.

Pacemakers or other biostimulators may be delivered to and retrieved from a patient using any suitable delivery and retrieval systems, such as those described in U.S. Pat. No. 10,856,905, issued on Dec. 8, 2020, entitled “Catheter-Based System for Delivery and Retrieval of a Leadless Pacemaker”, the disclosure of which is incorporated by reference in its entirety. In some implementations of delivery systems, pacemaker device 100 is attached or connected to a delivery system and advanced intravenously into the heart. The delivery system may include features to engage pacemaker 100 to enable fixation of the pacemaker device 100 to tissue. For example, in implementations where pacemaker device 100 includes a passive engaging mechanism, such as a one or more anchors, the delivery system may include a docking cap or key configured to engage the leadless pacemaker and apply torque to screw the active engaging mechanism into the tissue.

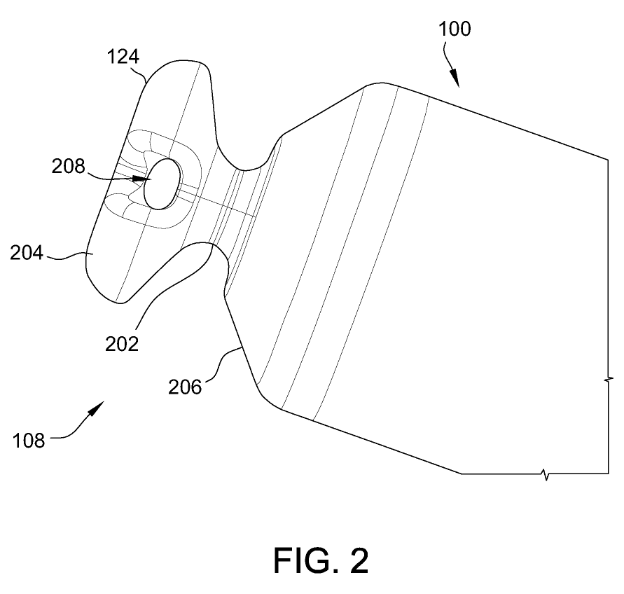

FIG. 2 is an enlarged view of proximal end 108 of pacemaker device 100, showing proximal attachment feature 124 (e.g., a button and stem). Proximal attachment feature 124 includes a stem 202, a button or head 204 that is flared radially outward relative to stem 202, and a hollow portion 208. The relatively narrow stem 202 and flared button 204 may cooperate to enable non-absorbing suture material to be tied around and held in place on stem 202 between flange 206 and the button 204 and/or tied through hollow portion 208. The suture material may then be used to anchor (e.g., implant) pacemaker device 100 at the desired location or to retrieve pacemaker device 100.

With the foregoing description of pacemaker technology set forth, the description will now proceed with examples of pacemaker devices in accordance with the present disclosure. The foregoing discussion is intended to provide information on pacemaker technology to facilitate a better understanding of the various aspects of the present disclosure, and is not intended to limit the scope of the present disclosure in any respect.

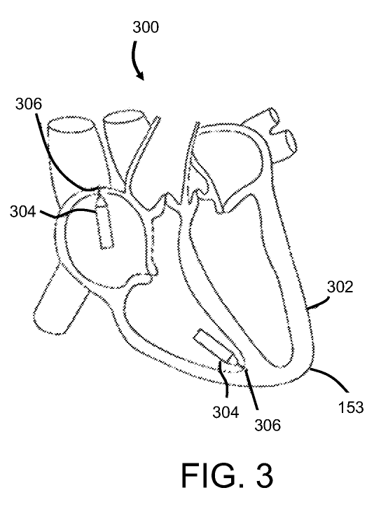

FIG. 3 illustrates a diagrammatic medial-lateral cross section of a patient’s heart 302 illustrating a pacing system 300 implanted in patient’s heart 302. Pacing system 300 includes one or more pacemaker devices 304, similar to pacemaker device 100 shown in FIG. 1). Each pacemaker device 304 is substantially enclosed in a housing (such as housing 151 shown in FIG. 1) suitable for placement on or attachment to the inside or outside of a cardiac chamber, such as the right/left atrium and/or right/left ventricle of patient heart 302, using a fixation mechanism 306. The housing defines a sealed cavity (such as sealed cavity 158 schematically depicted by dashed lines in FIG. 1). The sealed cavity is sized and shaped for containing battery material for powering pacemaker device 304 and pacing, sensing, and communication electronics and control circuitry. Attachment of pacemaker device 304 to the cardiac tissue may be accomplished via a distal end portion (such as distal end portion 130 shown in FIG. 1) which includes fixation mechanism 306 having one or more anchors on a mount (such as mount 155 shown in FIG. 1) extending distally from the distal end portion of the pacemaker device 304 and a distal electrode of the pacemaker device 304 (such as electrode 154 shown in FIG. 1). In the description that follows for pacemaker devices, the devices are placed or implanted within the one of the chambers of the heart 300.

### B. Example Pacemaker Devices With Unique Fixation Elements

In the examples that follow, pacemaker devices are described that include a distal end portion of the pacemaker device with a mount that attaches and provides sensing and/or pacing at a target site. The mount includes one or more fixation elements for anchoring the pacemaker device at the implantation site and one or more distal electrodes for sensing and/or pacing at the implantation site. The fixation elements may be passive (i.e., do not operate as electrodes) or active (i.e., operate as electrodes). The pacemaker device houses the electronics and circuitry for electrical signals delivered to and/or received from the electrodes. Advantageously, the pacemaker device is compact with a relatively small footprint, which increases the versatility of the pacemaker device with respect to locations where it may be implanted. For example, the pacemaker device may be sized and shaped for implanting at a site proximate within the patient’s heart (e.g., within one of the chambers of the heart). This in turn enables for an efficient and easier implantation process of the pacemaker device. Other advantages are described elsewhere herein and/or will become apparent upon reading the following description.

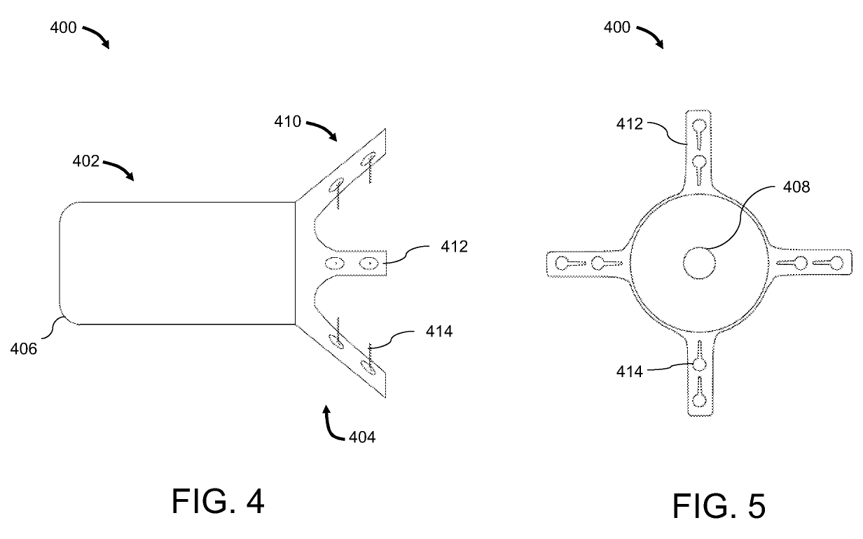

FIG. 4 illustrates an example of a pacemaker device 400 that includes a proximal end portion 402 and a distal end portion 404. Pacemaker device 400 may also be referred to as biostimulator or an implantable pulse generator (or an IPG). Pacemaker device 400 includes the features and components of pacemaker device 100 shown in FIGS. 1 and 2, with like reference numerals indicating like features and elements. The above description of pacemaker device 100 applies to pacemaker device 400 unless expressly stated otherwise or the context clearly indicates otherwise.

Similar to housing 151 described above for pacemaker device 100, a housing 406 of pacemaker device 400 may include a conductive material such as titanium, 316L stainless steel, or other similar materials, and is partially coated with an insulating or dielectric (e.g., polymeric) coating, with the uncoated region of housing 406 defining electrode 156 (e.g., a ring electrode) as shown in FIG. 1.

As may be understood from FIGS. 4 and 5, pacemaker device 400 also includes a header assembly, similar to that of header assembly 110 shown in FIG. 1, that may be mounted or attached (e.g., welded, threaded, or adhered) to housing 406, like the header assembly of pacemaker device 100, at a distal end portion 404 of pacemaker device 400. The header assembly may include an electrical feedthrough assembly including an electrical feedthrough (not shown). In the example pacemaker device 400, the header assembly includes a mount (not shown), similar to that of mount 155 as shown in FIG. 1, mounted on the electrical feedthrough assembly along the distal end portion 404. A distal electrode 408, similar to that of distal electrode 154 shown FIG. 1, may be located at the tip of the header assembly.

Pacemaker device 400 further includes a pliable assembly 410 is operably fixed on the mount at distal end portion 404. Pliable assembly 410 may be flexible (e.g., expandable/retractable) and may be made of polymer material, nitinol, titanium, 316L stainless steel, or other similar materials that enable pliable assembly 410 to function as described herein. Pliable assembly 410 may include one or more arms 412 (e.g., one or more tines 412) that may also be flexible and may extend outwardly from the distal end 404 to contact a patient’s tissue. The number of tines 412 may be adjusted to enable proper fixation in the patient. The tines 412 include hollow portions (e.g., holes or slots) and are operable to receive a fixation element. The fixation element is fixed to tines 412. Tines 412 operate to anchor the distal end of pacemaker device 400 in tissue (e.g., cardiac tissue) to sense and/or deliver electrical signals. Tines 412 may include materials such as nitinol, silicone rubber, such as titanium, 316L stainless steel, or other similar materials that enable the tines to function as described herein.

The fixation element may be in the form of at least one or more anchors 414. Anchors 414 may be passive fixation elements, meaning that it is relatively passive as the engagement of the anchors to the surface do not involve a screwing action, like a helical screw fixation element would. Anchors 414 operate to anchor distal end 404 of pacemaker device 400 in tissue (e.g., cardiac tissue) to sense and/or deliver electrical signals. In other examples, an active fixation element, may additionally or alternatively be included. Anchors 414 may include materials such as implantable plastic polymers, polyether ether ketone (PEEK), silicone rubber, nitinol, titanium, 316L stainless steel, or other similar materials that enable anchors 414 to function as described herein. The number of anchors 414 may be adjusted to enable proper fixation in the patient. In some cases, an active fixation element, such as a helical screw, may additionally or alternatively be included on distal end portion 404.

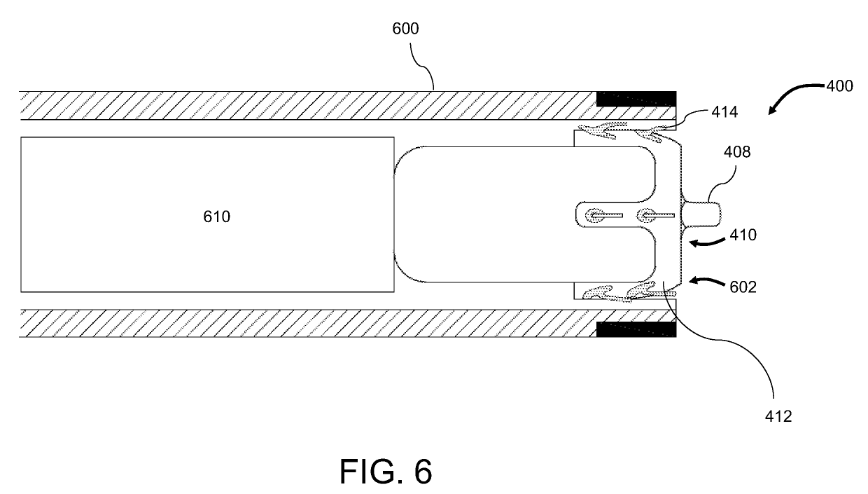

FIG. 6 is a side view of pacemaker device 400 prepared for implantation by being placed in a catheter 600. As shown, arms 412 and anchors 414 are positioned in catheter 600 in a direction away from a catheter opening 602. In some cases, arms 412 and anchors 414 are positioned in a direction towards catheter opening 602. Catheter 600 may include a push rod 610 that is operable to provide rigidity to catheter 600 and push pacemaker device 400 through catheter 600 while pacemaker device 400 is implanted into the target tissue. Distal electrode 408 may be used to detect the desired implantation target site of pacemaker device 400. Distal electrode 408 may be also used transmit and/or receive signals/information, such as described above with respect to distal electrode 154 shown in FIG. 1.

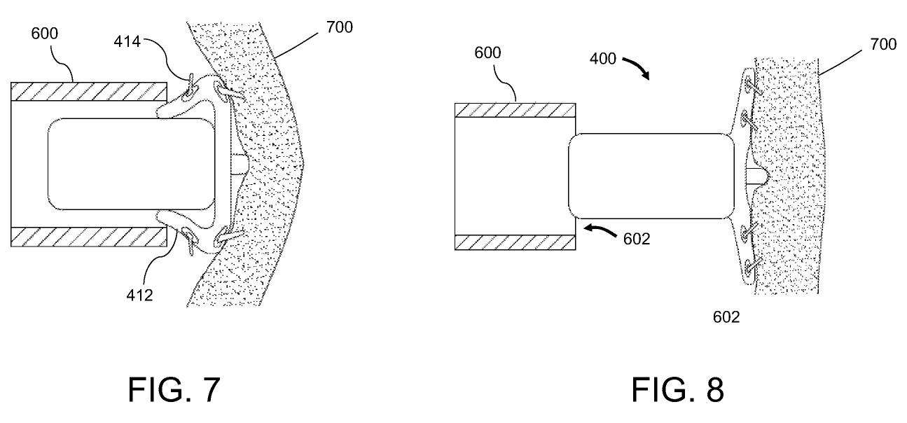

As may be understood from FIGS. 7 and 8, once catheter 600 is placed near the target cardiac tissue, pacemaker device 400 is released from catheter 600 through catheter opening 602. When pacemaker device 400 is released from catheter 600 arms 412 extend outwardly (as seen in FIG. 4) in such an operative position to embed anchors 414 in the target tissue and remain embedded. It is envisioned in some implementations, when arms 412 are made from silicone material, the pliable nature of the material will cause arms 412 to have potential energy when arms 412 are placed into catheter 600 in a direction away from catheter opening 602. When pacemaker device 400 is released from catheter 600, the potential energy will cause arms 412 to extend outwardly and embed anchors 414 in a direction to pull tissue 700 closer to electrode 408. Examples for well-known delivery methods are described, for example, in U.S. Pat. No. 10,179,236, issued on Jan. 15, 2019, entitled “Leadless Cardiac Pacing Devices,” the disclosure of which is incorporated by reference in its entirety. Once pacemaker device 400 has been embedded/implanted into the target tissue, catheter 600 may be retracted from the patient.

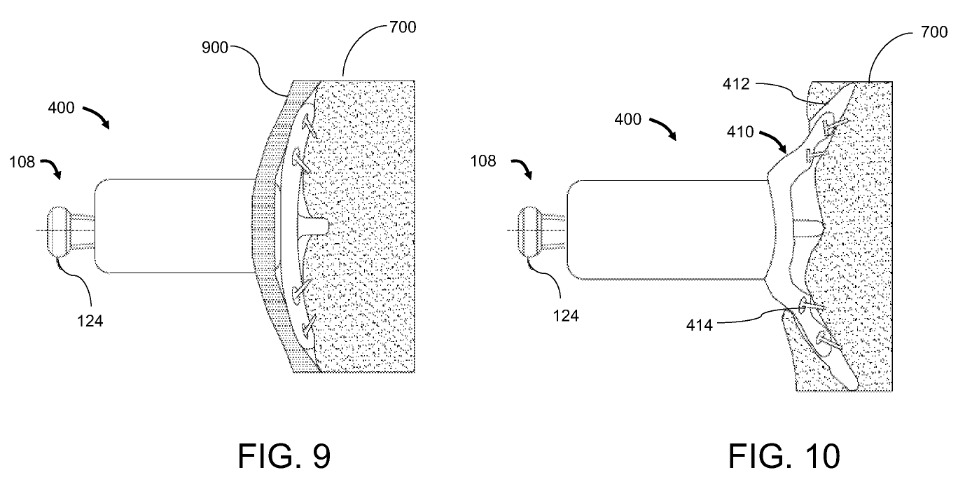

As may be understood from FIGS. 9 and 10, pacemaker device 400 may be removed from tissue 700 in a retrieval process. After pacemaker device 400 is implanted, the immune system of a patient may cause distal end 404 of pacemaker device 400 to be encapsulated by tissue 900 decreasing functionality and performance of pacemaker device 400. Pacemaker device 400 may be removed from the target tissue by pulling pacemaker device 400 in a direction away from tissue 700 and/or 900, such as in a lead extraction procedure.

As shown in FIGS. 9 and 10, pacemaker device 400 may include a proximal end portion 108 that may include a proximal attachment feature 124 (e.g., a button and stem, shown in dotted lines) as described above. Proximal attachment feature 124 includes a stem, a button or head that is flared radially outward relative to stem, and a hollow portion (as shown in FIG. 2). To retrieve pacemaker device 400 from a patient, a retrieval catheter is inserted into the patient and engages (e.g., via tethers) with proximal attachment feature 124 on proximal end 108 of pacemaker device 400. Once the tethers are sufficiently engaged, the tethers can be pulled in a direction away from the tissue 700 and/or 900, to disengage arms 412 and anchors 414. As shown in FIG. 10, it is envisioned that when pacemaker device 400 is pulled in a direction away from tissue 700 and/or 900, arms 412 of pliable assembly 410 stretch and enable anchors 414 to pivot or change direction in tissue 700 for easier removal. Once anchors 414 are retrieved from tissue 700 and 900, the retrieval catheter and pacemaker device 400 may be removed from the patient. Additionally, retrieval of pacemaker device 400 may be implemented using or one or more methods and systems described herein with respect to pacemaker device 100.

The fixation elements and electrodes of the pacemaker devices described herein may be used in isolation or in any combination. Thus, any of one or more electrodes, fixation elements or features described herein may be used in combination with other one or more electrodes, fixation element, or features described herein. Additionally, in cases where two or more fixation elements or two or more distal electrodes are used in combination, one of the fixation elements and one of the distal electrodes may also be used in isolation.

Although certain examples of this disclosure have been described above with a certain degree of particularity, those skilled in the art could make numerous alterations to the disclosed implementations without departing from the spirit or scope of this disclosure. All directional references (e.g., plus, minus, upper, lower, upward, downward, left, right, leftward, rightward, top, bottom, above, below, vertical, horizontal, clockwise, and counterclockwise) are only used for identification purposes to aid the reader’s understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

As for additional details pertinent to the present disclosure, materials and manufacturing techniques may be employed as within the level of those with skill in the relevant art. The same may hold true with respect to method-based aspects of the disclosure in terms of additional acts commonly or logically employed. Also, it is contemplated that any optional feature of the variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this disclosure belongs.

What is claimed is:

1. A pacemaker device comprising:
a proximal end portion comprising a housing defining a cavity for containing pacing electronics and battery material;
a mount positioned at a distal end of the pacemaker device;
a pliable assembly coupled to the mount; and
one or more electrodes electrically configured to perform at least one of receiving one or more electrical signals from a target tissue site or delivering one or more pacing signals to the target tissue site.

2. The pacemaker device of claim 1, wherein the pliable assembly comprises one or more arms.

3. The pacemaker device of claim 2, wherein the one or more arms comprise a plurality of slots operable to receive one or more anchors.

4. The pacemaker device of claim 2, wherein the one or more arms are flexible and extend outwardly to contact the target tissue site.

5. The pacemaker device of claim 2, wherein a number of the one or more arms is adjustable to enable adequate fixation of the pacemaker device in the target tissue.

6. The pacemaker device of claim 1, wherein the pliable assembly comprises one or more materials being at least one of implantable plastic polymers, polyether ether ketone (PEEK), silicone rubber, nitinol, titanium, or 316L stainless steel.

7. The pacemaker device of claim 1, wherein the pliable assembly comprises at least one fixation element operable to be in contact with the target tissue.

8. The pacemaker device of claim 7, wherein the at least one fixation element is at least one of a passive fixation element or an active fixation element.

9. The pacemaker of claim 1, wherein the pliable assembly comprises the one or more electrodes.

10. The pacemaker of claim 1, wherein the one or more electrodes are operable to be in contact with the target tissue.

11. A method for positioning a pacemaker device into a target tissue site, the method comprising:
guiding the pacemaker device to the target tissue site, wherein the pacemaker device includes a mount and a pliable assembly coupled to the mount, and wherein the pliable assembly includes one or more anchors;
in response to finding the target tissue site, expanding the pliable assembly to contact the target tissue site;
inserting the one or more anchors into the target tissue site; and
delivering to the target tissue site, using the pliable assembly, one or more pacing signals.

12. The method of claim 11 further comprising receiving from the target tissue site, using the pliable assembly, one or more electrical signals.

13. The method of claim 11 further comprising positioning, into the target tissue site, a distal electrode coupled to the mount.

14. The method of claim 13 further comprising transmitting to the target tissue site, using the distal electrode, the one or more pacing signals.

15. The method of claim 13 further comprising receiving from the target tissue site, using the distal electrode, one or more electrical signals.

16. The method of claim 13, wherein the pliable assembly includes one or more arms.

17. The method of claim 16, wherein the one or more arms include the one or more anchors.

18. The method of claim 11, wherein the pliable assembly includes one or more materials being at least one of implantable plastic polymers, polyether ether ketone (PEEK), silicone rubber, nitinol, titanium, or 316L stainless steel.

19. The method of claim 11 further comprising positioning, into the target tissue site, least one fixation element operable to be in attach the pacemaker device to the target tissue.

20. The pacemaker device of claim 19, wherein the at least one fixation element is at least one of a passive fixation element or an active fixation element.
